(19) United States
(12) Patent Application Publication
(10) Pub. No.: US 2014/0364945 A1
(43) Pub. Date: Dec. 11, 2014

(54) ANNULOPLASTY DEVICE

(71) Applicant: LC Therapeutics, Inc., Fremont, CA (US)

(72) Inventors: James Longoria, Sacramento, CA (US); Roy Chin, Pleasanton, CA (US)

(21) Appl. No.: 14/296,336

(22) Filed: Jun. 4, 2014

Related U.S. Application Data
(60) Provisional application No. 61/831,450, filed on Jun. 5, 2013.

(51) Int. Cl. A61F 2/24

(52) U.S. Cl. CPC A61F 2/2445
USPC 623/2.36

(57) ABSTRACT

Annuloplasty devices and methods for using the same are provided. Aspects of the devices include an at least partially annular body having at least one integrated tissue securing region. Also provided are methods of implanting the devices, as well kits for practicing the same. The devices, kits and methods find use in a variety of different applications, including cardiac valve repair applications.

### CROSS-REFERENCE TO RELATED APPLICATIONS

Pursuant to 35 U.S.C. §119 (e), this application claims priority to the filing date of U.S. Provisional Patent Application Ser. No. 61/831,450 filed Jun. 5, 2013; the disclosure of which application is herein incorporated by reference.

### INTRODUCTION

In humans and other vertebrate animals, the heart is a hollow muscular organ having four pumping chambers separated by four heart valves: aortic, mitral (or bicuspid), tricuspid, and pulmonary. During the cardiac cycle of contraction and relaxation, the valves open and close in response to a pressure gradient to control the flow of blood to a particular region of the heart and/or to blood vessels (pulmonary aorta, etc.)

Cardiac valves include a dense fibrous ring known as the annulus, and leaflets or cusps attached to the annulus. For some valves, e.g., the mitral valve, there is also a complex of chordae tendinae and papillary muscles securing the leaflets. The size of the leaflets or cusps is such that when the heart contracts the resulting increased blood pressure formed within heart chamber forces the leaflets open to allow flow from the heart chamber. As the pressure in the heart chamber subsides, the pressure in the subsequent chamber or blood vessel becomes dominant, and presses back against the leaflets. As a result, the leaflets or cusps come in apposition to each other, thereby closing the passage.

Heart valve disease is a widespread condition in which one or more of the valves of the heart fails to function properly. Diseased heart valves may be categorized as either stenotic, wherein the valve does not open sufficiently to allow adequate forward flow of blood through the valve, and/or incompetent, wherein the valve does not close completely, causing excessive backward flow of blood through the valve when the valve is closed, which is also known as regurgitation. Valve disease can be severely debilitating and even fatal if left untreated. Various surgical techniques may be used to repair a diseased or damaged valve. In a traditional valve replacement operation, the damaged leaflets are typically excised and the annulus sculpted to receive a replacement prosthetic valve.

In patients who suffer from dysfunction of the mitral and/or tricuspid valve(s) of the heart, surgical repair of the valve (i.e., “valvuloplasty”) is a desirable alternative to valve replacement. Remodeling of the valve annulus (i.e., “annuloplasty”) is central to many reconstructive valvuloplasty procedures. In annuloplasty protocols, a prosthetic ring (i.e., “annuloplasty ring”) is implanted to stabilize the annulus and to correct or prevent valvular insufficiency that may result from defect dysfunction of the valve annulus. The annuloplasty ring is designed to support the functional changes that occur during the cardiac cycle: maintaining coaptation and valve integrity to prevent reverse flow while permitting good hemodynamics during forward flow. Annuloplasty procedures are performed not only to repair damaged or diseased annuli, but also in conjunction with other procedures, such as leaflet repair.

### SUMMARY

Annuloplasty devices and methods for using the same are provided. Aspects of the devices include an at least partially annular body having at least one integrated tissue securing region. Also provided are methods of implanting the devices, as well kits for practicing the same. The devices, kits and methods find use in a variety of different applications, including cardiac valve repair applications.

### BRIEF DESCRIPTION OF THE FIGURES

FIG. 1 is a diagram of an annuloplasty device having a full ring shape including tissue securing regions according to embodiments of the present disclosure. The diagram of FIG. 1 also shows tissue securing devices according to embodiments of the present disclosure.

FIG. 2 is a diagram providing a side-view of an annuloplasty device having a full ring shape according to embodiments of the present disclosure.

FIG. 3A is a diagram providing a cross-sectional view of an annuloplasty device including a tissue securing region according to embodiments of the present disclosure. The diagram of FIG. 3A also shows a tissue securing device according to embodiments of the present disclosure. FIG. 3B is a diagram providing a cross-sectional view of an annuloplasty device according to embodiments of the present disclosure. FIGS. 3C to 3E provide additional views of a device according to the embodiment shown in FIGS. 3A and 3B.

FIG. 4 is a diagram of an annuloplasty device having a partial ring shape including tissue securing regions according to embodiments of the present disclosure.

FIG. 5 is a diagram providing a perspective view of an annuloplasty device having a partial ring shape including tissue securing regions according to embodiments of the present disclosure.

FIG. 6A is a diagram providing a top view of a saddle shaped annuloplasty device according to embodiments of the present disclosure.

FIG. 6B is a diagram providing a view of a saddle shaped annuloplasty device taken in direction X according to embodiments of the present disclosure.

FIG. 6C is a diagram providing a view of a saddle shaped annuloplasty device taken in direction Y according to embodiments of the present disclosure.

FIG. 7A provides an illustration of a cross sectional view of the left ventricle of a heart including an exposed mitral valve according to embodiments of the present disclosure.

FIG. 7B is a diagram illustrating an annuloplasty device including tissue securing regions and seated on the annulus of a mitral valve according to embodiments of the present disclosure.

FIG. 7C is a diagram illustrating an annuloplasty device including tissue securing regions being affixed to the annulus of a mitral valve according to embodiments of the present disclosure.

FIG. 7D is a diagram illustrating an annuloplasty device having tissue securing devices deployed through each of its tissue securing regions according to embodiments of the present disclosure.

### DEFINITIONS

The term “annuloplasty”, as described herein, refers to a surgical procedure in which a heart valve (e.g., mitral valve) annulus is remodeled. Remodeling a heart valve may include, for example, reinforcing and/or re-shaping the valve by implanting a prosthetic ring (e.g., an annuloplasty ring) to stabilize the annulus and to correct or prevent valvular insufficiency.

Any of the embodiments of the disclosed annuloplasty devices may be configured (e.g., sized and/or shaped) to be implanted at a tissue site, such as a cardiac valve site (e.g., a mitral valve site). As used herein, the phrase “cardiac valve site” refers to the location within the body of a subject at, contacting, within, above, below and/or immediately adjacent to (e.g., within 1, 2, 3, 4, 5, 6, 7, 8, 9, or 10 mm of) one or more cardiac valves (e.g., the mitral, tricuspid, aortic and/or pulmonary valves). Accordingly, the phrase “mitral valve site” refers to the location within the body of a subject at, contacting, within, above, below and/or immediately adjacent to the mitral valve.

As noted above, annuloplasty may include securing (e.g., affixing, fastening or attaching for a period of time such as for at least the remaining lifetime of a subject) an annuloplasty device to one or more tissues using a tissue securing device. By “securing” is meant stably associating the device with a tissue location, such that the device and tissue location do not separate from each other under normal physiologic conditions. As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, nervous tissue and/or epithelial tissue.

The term “subject” is used interchangeably herein with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform an annuloplasty procedure on a human subject, it is to be understood that the subject devices and methods may also be carried-out to perform an annuloplasty procedure on other subjects (that is, in “non-human subjects”).

The present disclosure provides embodiments of devices (e.g., annuloplasty devices) which are implantable. As used herein, the terms “implantable”, “implanted” and “implanting” refer or relate to the characteristic of the ability of an aspect to be placed (e.g., surgically introduced) into a physiological site (e.g., a site within the body of a subject) and maintained for a period of time without substantial, if any, impairment of function. As such, once implanted in or on a body, the aspects do not deteriorate in terms of function, e.g., as determined by ability to perform effectively as described herein, for a period of 2 days or more, such as 1 week or more, 4 weeks or more, 6 months or more, or 1 year or more, e.g., 5 years or more, and/or for the remaining lifetime or expected remaining lifetime of the subject or more. Implantable aspects may also be aspects that are configured (e.g., dimensioned and/or shaped) to fit into a physiological site (e.g., a site within the body of a subject). For example, in certain embodiments, an implantable aspect may have a longest dimension, e.g., length, width or height, ranging from 0.05 mm to 50 mm, such as from 0.2 mm to 25 mm, including from 0.5 mm to 20 mm. Implanting may also include securing an implanted object (e.g., a prosthetic device) to one or more tissues within the body of the subject. Additionally, implanting may, in some instances, include all of the surgical procedures (e.g., cutting, suturing, sterilizing, etc.) necessary to introduce one or more objects into the body of a subject.

The devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue piercing device enters tissue). Similarly, the term “distal” refers to a direction oriented away from the operator during use or a position (e.g., a spatial position) further from the operator (e.g., closer to a subject or tissue thereof) during use (e.g., at a time when a tissue piercing device enters tissue). Accordingly, the phrase “proximal end” refers to that end of the device that is closest to the operator during use, while the phrase “distal end” refers to that end of the device that is most distant to the operator during use.

Geometrical terms are also used to describe the disclosed devices throughout the specification. As such, the term “plane” as used herein refers to a flat surface that is infinitely large with zero thickness, unless a particular thickness is otherwise specified or can reasonably be inferred. Additionally, the term “longitudinal” as used herein refers to the characteristic of being associated with (e.g., placed or running along) a length (e.g., a straight or curved length) or lengthwise dimension.

Furthermore, the definitions and descriptions provided in one or more (e.g., one, two, or three, etc.) sections of this disclosure (e.g., the “Descriptions”, “Devices”, “Methods” and/or “Kits” sections below) are equally applicable to the devices, methods and aspects described in the other sections.

### DETAILED DESCRIPTION

Annuloplasty devices and methods for using the same are provided. Aspects of the devices include an at least partially annular body having at least one integrated tissue securing region. Also provided are methods of implanting the devices, as well kits for practicing the same. The devices, kits and methods find use in a variety of different applications, including cardiac valve repair applications.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Devices

As summarized above, aspects of the invention include implantable annuloplasty devices having one or more tissue securing regions each configured to receive a tissue securing device. Aspects of the invention further include tissue securing devices configured to secure annuloplasty devices within the body of a subject. Each of these aspects of the invention is now described further in greater detail.

### Annuloplasty Devices

Embodiments of the disclosed devices include annuloplasty devices having a body (e.g., an at least partially annular body) and one or more tissue securing regions. Tissue securing regions of annuloplasty devices are regions of the devices configured for use in attaching the annuloplasty device to tissue of a subject. Attaching an annuloplasty device to tissue of a subject may include securing (e.g., affixing, fastening, anchoring or attaching) the annuloplasty device to the tissue for a period of time (e.g., for the remaining lifetime of a subject or for a portion of the remaining expected lifetime of a subject).

As noted above, annuloplasty devices may have one or more (e.g., two or more; three or more; four or more; or five or more) tissue securing regions. For example, an annuloplasty device may have one or between one and one-hundred one (e.g., 2; 3; 4; 5; 6; 7; 8; 9; 10; 11; 12; 13; 14; 15; 16; 17; 18; 19; 20; 21; 22; 23; 24; 25; 26; 27; 28; 29; 30; 31; 32; 33; 35; 36; 37; 38; 39; 40; 41; 42; 43; 44; 45; 46; 47; 48; 49; 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100) tissue securing regions. In some embodiments, annuloplasty devices may have 10 or less; 14 or less; 15 or less; 20 or less; 25 or less; 30 or less; 50 or less; or 100 or less tissue securing regions. In some embodiments, the number of tissue securing regions on an annuloplasty device may range from 1-5; 1-10; 1-15; 1-20; 1-25; 1-30; 1-50; 1-100; 5-15; 5-20; 5-25; 5-30; 10-15; 10-25; 10-30 or 10-40. In some embodiments, the number of tissue securing regions of an annuloplasty device may be even or odd.

In various aspects, tissue securing regions of annuloplasty devices are integrated into the body of the annuloplasty devices. By “integrated” is meant located within, surrounded by, composed at least partially of the same material, and/or having a continuous shape with. For example, in some aspects, tissue securing regions may include one or more voids in the annuloplasty device (e.g., holes through the body of an annuloplasty device). In some aspects, one or more tissue securing regions may be fully or partially contained within (e.g., positioned between two portions of) the body of an annuloplasty device. Tissue securing regions or portions (e.g., first and second ends, such as opposite ends) of tissue securing regions of annuloplasty devices may also follow the same general contour of the body of the annuloplasty device. In these instances, two or more surfaces or openings (e.g., the top and bottom surface or opposite openings of a tissue securing region may be flush with and/or not protrude above the adjacent surfaces of the annuloplasty device). Tissue securing regions may also have any of the volumes and/or other dimensions disclosed herein. Furthermore, and as is discussed further detail below, embodiments of tissue securing regions may include a void and/or may be filled with one or more materials, such as one or more of the same materials as the body of an annuloplasty device.

In certain aspects of the disclosed devices, tissue securing regions are spaced apart (e.g., separated by a distance or positioned in different locations) on an annuloplasty device (e.g., on the body of an annuloplasty device). For example, one tissue securing region may be separated from another tissue securing region of an annuloplasty device by a portion of the annuloplasty device such as the body of the annuloplasty device or one or more materials thereof. Tissue securing regions may be spaced from each other along the body of an annuloplasty device by, for example, a distance in the range 0.5 mm to 50 mm, such as 1 mm to 15 mm, including 1 mm to 10 mm, e.g., 5 mm to 10 mm. In some versions, tissue securing regions are equidistantly spaced apart from each other. In versions of the devices having three or more tissue securing regions which are equidistantly spaced apart from each other, each tissue securing region may be separated by an equal distance (e.g., distance along the circumference and/or the body of the annuloplasty device) from the two tissue securing regions located spatially closest and/or adjacent to the tissue securing region. In versions of the devices having three or more tissue securing regions which are equidistantly spaced apart from each other, each tissue securing region may have an equal amount of the material of the body of the annuloplasty device positioned between the tissue securing region and the two tissue securing regions located spatially closest and/or adjacent to the tissue securing region. In some versions of the devices having three or more tissue securing regions, some of the tissue securing regions are spaced equidistantly apart from each other and other tissue securing regions are not equidistantly spaced apart (e.g., separated by an equal distance from the two tissue securing regions located spatially closest and/or are adjacent to the tissue securing region) from each other. In some instances, the tissue securing regions are not configured so as to provide for a differentiated flexibility in dependence on the location and direction of application of external stress experienced by the device, e.g., as described in U.S. Pat. No. 7,220,277.

The disclosed devices, in various embodiments, include one or more tissue securing regions which include a void in an annuloplasty device (e.g., a hole or channel through the body of an annuloplasty device). Where a tissue securing region includes a void, the void may be entirely contained within or positioned between at least two or more portions of an annuloplasty device (e.g., the body of an annuloplasty device). Where a tissue securing region includes a void, the void may be exposed to the environment surrounding the annuloplasty device and as such, may be filled with a substance from the surrounding environment such as a gas and/or liquid (e.g., air and/or water and/or blood) during use. Where a tissue securing region includes a void, the void may be configured (e.g., sized and/or shaped) to receive a tissue securing device or a portion of a tissue securing device therein or there-through. In some devices of tissue securing regions (e.g., tissue securing regions where a tissue securing region includes a void), the tissue securing region or void may be defined by one or more (e.g., one, two, three, or four) surfaces (e.g., a tubular surface) of the body of an annuloplasty device. As such, in some embodiments, tissue securing regions include one or more (e.g., one, two, three, or four) surfaces (e.g., a tubular surface) immediately surrounding (e.g., adjacent to), lining, or defining one or more voids or holes in an annuloplasty device. In some embodiments of devices in which tissue securing regions include or are defined by two or more surfaces of an annuloplasty device or portion thereof (e.g., an annuloplasty device body), the surfaces may be separated from one another by one or more edges (e.g., one, two, three, four, five, or six edges), such as edges that are equidistantly spaced from each other.

In various aspects, the disclosed devices may include one or more tissue securing regions which include one or more (e.g., one, two, three, or four) different materials from the one or more materials of the body of an annuloplasty device. For example, the disclosed devices may include one or more tissue securing regions which include one or more (e.g., one, two, three, or four) materials that are more compliant (e.g., more malleable, softer, less rigid, less dense, more pliable and/or more readily pierced by an object such as a tissue securing device) than the one or more materials of the body of an annuloplasty device. Where a tissue securing region includes one or more compliant materials, a portion (e.g., one, two, or more surfaces) of the material of the tissue securing region may be exposed to the environment surrounding the annuloplasty device and may seal the tissue securing region and thereby prevent the tissue securing region from becoming filled with a substance from the surrounding environment such as a gas and/or liquid (e.g., air and/or water and/or blood). Where a tissue securing region includes one or more different, e.g., compliant, materials and a portion (e.g., one, two, or more surfaces) of the material of the tissue securing region is exposed to the environment surrounding the annuloplasty device, the one or more exposed portions may generally conform to the shape of the annuloplasty device body. In certain embodiments, a first portion of a tissue securing region includes a compliant material and a second portion of the same tissue securing region includes a void. Additionally, the subject devices may include one or more tissue securing regions which include one or more (e.g., one, two, three, or four) materials that are the same as (e.g., the same type and/or one continuous piece with) the one or more materials of the body of an annuloplasty device.

In some embodiments of the disclosed devices, one or more (e.g., one, two, three, or four) materials of one or more tissue securing regions may be any suitable material, where materials of interest include biocompatible materials, which materials may or may not be biodegradable. Specific materials of interest include, but are not limited to: polymeric materials, e.g., plastics, rubbers, silicones, etc.

Embodiments of the disclosed annuloplasty devices include one or more tissue securing regions, wherein each tissue securing region may have a wide variety of shapes and sizes. The shapes and/or sizes of all of the tissue securing regions in an annuloplasty device may be the same or the shapes and/or sizes of the tissue securing regions in a device may be different from one another.

The disclosed devices, in certain variations, include one or more tissue securing regions (e.g., tissue securing regions which include a void in an annuloplasty device, such as a hole or channel through the body of an annuloplasty device, and/or which include one or more materials, such as a compliant material) that have a shape (e.g., a tubular shape or a nontubular shape) defined, for example, by one or more surfaces of the body of an annuloplasty device.

Embodiments of the disclosed devices include one or more tissue securing regions that are sized and/or shaped to receive and/or retain one or more tissue securing devices. Tissue securing devices which may be received and/or retained by the tissue securing regions are described in further detail below.

In some instances, tissue securing regions (e.g., tissue securing regions forming a hole, channel, or aperture through an annuloplasty device body) have a shape that is defined by a first opening (e.g., a first opening in the body of an annuloplasty device body) and a second opening (e.g., a second opening in the body of an annuloplasty device body). The first and second openings may have any suitable size or shape (e.g., circle, semi-circle, oval, rectangle, square, triangle, polygon, quadrilateral, or combination thereof). The first and second openings may also have the same size and/or shape or a different size and/or shape. The first and second openings may also be continuous or flush with the portion of the body of the annuloplasty device adjacent to or defining the openings.

In certain embodiments, tissue securing regions each have a single longitudinal axis (e.g. an axis of symmetry) passing (e.g., passing centrally through the tissue securing region) from a first end (e.g., the center-most point of the first end) of a tissue securing region to a second end (e.g., the center-most point of the second end) of the tissue securing region. In some versions, the shape of a tissue securing regions is symmetrical about the longitudinal axis. In some variations, the shape of tissue securing regions (e.g., a square or rectangular tissue securing region) is symmetrical about at least one plane (e.g., one, two, three, or four planes) that includes the longitudinal axis of the tissue securing region. Embodiments of the to disclosed devices include devices in which the longitudinal axis of one or more (e.g., all) tissue securing regions of a device are parallel to one another. Aspects of the disclosed devices also include devices in which the longitudinal axes of two or more tissue securing regions of a device are not all parallel to one another. Disclosed devices include those which include a first set of two or more tissue securing devices in which the longitudinal axis of each tissue securing region of the first set are parallel to one another and a second set of two or more tissue securing devices in which the longitudinal axis of each tissue securing region of the second set are parallel to one another but are not parallel to the longitudinal axes of the tissue securing regions of the first set.

In some embodiments of tissue securing regions having a shape defined by a first opening and a second opening, the first opening and second opening are connected to one another by a channel or hole (e.g., a channel defined by one or more surfaces of the body of an annuloplasty device) through the body of an annuloplasty device. The channel may be shaped substantially as a cylinder having a single radius about a central axis (e.g., an axis of symmetry). The channel may also be contained entirely within the body of an annuloplasty device (e.g., be positioned between at least two portions of the body of an annuloplasty device). In some embodiments, the channel connecting the first and second opening has a radius that is larger or smaller at the first opening compared to that at the second opening. In some embodiments, the channel connecting the first and second opening has a radius that is larger or smaller at the first opening and/or the second opening compared with that at a position along a central axis (e.g., an axis of symmetry) of the channel between the first and second opening.

As noted above, in various embodiments, the tissue securing region has a tubular shape. In aspects of tissue securing regions having a tubular shape, the tissue securing region may be defined by a first and second opening, each having a circular or ovoid shape (e.g., a circular or ovoid shape of the same size and shape) and connected by a channel within the body of an annuloplasty device. In versions of the device in which the tissue securing region has a tubular shape, the tissue securing region and/or the channel within the body has a longitudinal axis (e.g., an axis of symmetry) running centrally through the channel and about which the tissue securing region and/or channel may be symmetrical. Such a longitudinal axis may pass through the center of the first and second circular or ovoid openings. Additionally, in versions of the device in which the tissue securing region has a tubular shape, the radius of the first and second openings and any radius of a cross section of the channel between the openings (e.g., a cross section that is perpendicular to the longitudinal axis of the channel) may all have the same length.

As noted above, in some embodiments, the tissue securing region has a nontubular shape. In aspects of tissue securing regions having a nontubular shape, the tissue securing region may be defined by a first and second opening, each having a square, rectangular, triangular, or other matching or non-matching shape and connected by a channel within the body of an annuloplasty device. The channel may have a central longitudinal axis (e.g., an axis of symmetry) passing through the center-most point of the first and second openings. Additionally, in some versions of the device, the shape and/or size (e.g., the size of the area, perimeter or circumference) of the first and the second openings may be the same as that of any cross section or all cross sections of the channel between the openings (e.g., a cross section that is perpendicular to the longitudinal axis of the channel). In aspects of tissue securing regions having a nontubular shape, a tissue securing region may be symmetrical with respect to one or more planes (e.g., only one plane, or only two planes passing through the tissue securing region, such as one or two planes including the central longitudinal axis of a tissue securing region).

In some embodiments, annuloplasty devices or the bodies thereof have a top surface (e.g., a surface oriented upward when the device is placed on a surface and/or implanted into the body of a subject) and a bottom surface (e.g., a surface oriented downward when the device is placed on a surface and/or implanted into the body of a subject). As such, in some embodiments, one or more tissue securing regions or the longitudinal axis of the one or more tissue securing regions or the channels thereof extend vertically (i.e., in the vertical direction) through an annuloplasty device (e.g., from the top side through the bottom side). In some embodiments, one or more (e.g., two or more) tissue securing regions or the longitudinal axis of the one or more tissue securing regions or the channels thereof extend through an annuloplasty device or the body thereof at one or more angles (e.g., an angle ranging from 1° to 90°, such as 10°, 45°, 90°) from the vertical direction. In such embodiments, the one or more angles from the vertical direction at which a longitudinal axis extends may be within any of the cross-sectional planes of the annuloplasty devices described further below.

Embodiments of the disclosed devices include one or more tissue securing regions each having a volume suitable for receiving a tissue securing device, e.g., as described in greater detail below. While volumes of tissue securing regions may vary, in some instances the tissue securing regions have a volume ranging from 0.1 mm³ to 10 mm³, e.g., from 1 mm³ to 5 mm³, including from 1 mm³ to 2 mm³. Additionally, in some embodiments, tissue securing regions may have a dimension, (e.g., cross-sectional diameter, length, such as a longitudinal length, width or height), such as a longest dimension, ranging from 0.01 mm to 10 mm, such as from 1 mm to 5 mm, include from 1 mm to 2 mm. In some aspects of the disclosed devices, all the tissue securing regions of a device have the same size and/or shape and/or volume. In certain aspects, different tissue securing regions of a device have different sizes and/or shapes and/or volumes. In certain embodiments, tissue securing regions have a volume sufficient to receive and/or retain any of the tissue securing devices described herein.

In various aspects, the disclosed devices may include one or more tissue securing regions that are marked. Tissue securing regions may be marked and thereby made distinguishable (e.g., visually distinguishable) from other portions of annuloplasty devices (e.g., the annuloplasty device body) by having a different (e.g., contrasting) color (e.g., white, black, grey, red, blue, yellow, green, purple, orange, pink, or any combination or shade thereof, etc.) and/or texture than the other portions of the annuloplasty devices (e.g., the portion of the annuloplasty device body immediately adjacent to the tissue securing region). In some embodiments, tissue securing regions may be marked by being at least partially composed of a different color and/or texture of material than the remaining portions (e.g., the annuloplasty device body) of the annuloplasty device. In some embodiments, tissue securing regions may be marked by being at least partially composed of or including a first color (e.g., red or green) and/or texture of material wherein the remaining portions (e.g., the annuloplasty device body) of the annuloplasty device may at least partially be composed of or include a second color (e.g., blue or yellow). In certain aspects, tissue securing regions that are marked may have an associated marking shaped as any letter, number, shape (e.g., circle, semi-circle, oval, rectangle, square, triangle, polygon, quadrilateral), or combination thereof (e.g., a bar-code). In various aspects, tissue securing regions that are marked may have a marking that is a stripe of colored (e.g., black) material or substance on the annuloplasty device. In some versions of the devices, markings of tissue securing regions are fully or partially positioned on the annuloplasty device body.

In various aspects, tissue securing regions are marked by one or more electronic components (e.g., a circuit and/or circuit component and/or microchip) and/or magnetic substances. Electronic components or magnetic substances with which tissue securing regions can be marked may be any components or substances applicable in determining the position (e.g., proximity and/or orientation) of the component or substance in relation to another aspect, such as a sensing device. In some embodiments, markings of tissue securing regions are recognizable by one or more electronic components, such as a sensing device (e.g., a scanner). Such a sensing device may be incorporated (e.g., incorporated at the distal end) with a device for deploying a tissue securing device. Embodiments of possible sensing devices include an indicator (e.g., a visual display and/or an auditory signal generator) configured for indicating when the sensing device, such as a device for deploying a tissue securing device, is in a particular position (e.g., proximity and/or orientation) with respect to the tissue securing region, for example, a position such that a tissue securing device can effectively be deployed at the tissue securing region. In certain embodiments, a sensing device may also include or be configured to interact with (e.g., by one or more electrical connections or by a wireless signal) a control system (e.g., a computer) which may also be configured to send and/or display one or more signals for indicating a particular position (e.g., proximity and/or orientation) of the sensing device with respect to the tissue securing region.

As noted above, in addition to one or more tissue securing regions, annuloplasty devices include a body. The body of an annuloplasty device may include all portions of the device that are not the tissue securing regions of the device. As such, the body of the annuloplasty device may have the same general shape (e.g., may have the same shape as the annuloplasty device but for the tissue securing regions of the device) as the annuloplasty device. Accordingly, the description of the shape, size and/or materials of the annuloplasty device provided below may also be individually applicable to the body of the annuloplasty device.

Embodiments of the disclosed devices include annuloplasty devices having a wide variety of shapes and sizes. In some embodiments, annuloplasty devices are annular or have an annular portion. The term “annular” as used herein refers to the characteristic of forming or being shaped like a ring (e.g., shaped generally like a torus). An annular aspect may also be circular or ovoid and have a cross section at any or all portions along its length having any shape (e.g., circular, ovoid, rectangular, etc.). An annular aspect may have one or more (e.g., two, three, or four) peripheral edges that form a curve. An annular aspect, in certain aspects, can also form an ellipse and/or may have one or more (e.g., two, three, or four) peripheral edges having straight (i.e., non-curved) portions.

As such, in some versions, annuloplasty devices are shaped as a ring (e.g., a ring that is substantially circular or ovoid) and/or a circular or ovoid tube. Such a ring shape may be full (e.g., forming a continuous band of material) or partial (e.g., forming a portion of a continuous band of material to form, for example, half of a generally ovoid shape). Such a ring may also be planar or non-planar. Examples of annuloplasty devices shaped as a full ring having characteristics that may be utilized either wholly or partially in connection with the disclosed devices and methods are provided by U.S. Pat. Nos. 5,104,407, 4,489,446; 4,306,319; 4,290,151; 4,204,283; 4,055,861; 4,042,979; 3,656,185; 8,287,591; 8,353,956; 8,382,828; 8,216,304; 8,241,351; 8,142,495; 8,197,538; 6,102,945; 6,174,332; 6,143,024; 5,061,277; 7,361,190; 8,088,159; 8,142,495; 7,951,196; 7,959,673; 7,938,856; 7,993,395; 8,012,202; 7,842,085; 7,879,087; and 8,034,103, the disclosures of each which are incorporated by reference herein.

In certain versions, annuloplasty devices are shaped as a circular or ovoid portion (e.g., tube) of continuous material. Various embodiments of the disclosed devices include kidney-shaped or substantially kidney-shaped annuloplasty devices. In some versions, annuloplasty devices have one or more curved (e.g., curved along the longitudinal length) portions or one or more non-curved (i.e., straight) portions. In some embodiments, annuloplasty devices have one or more curved portions (e.g., curved along the longitudinal length) that define two or more radii of curvature lying within a plane (e.g., a horizontal plane). As such, the center-most line in an annuloplasty device may arc and/or form at least a portion of an ovoid or circular shape in a horizontal direction with respect to the top and bottom surfaces (or upper-most and bottom-most portions) of a device. In some embodiments, the center-most line in the body of an annuloplasty device passes through one or more (e.g., all) of the tissue securing regions of the device. In some embodiments, annuloplasty devices (e.g., annuloplasty devices that are annular or have an annular portion) have a longitudinal body or portion thereof (e.g., a portion of a device extending in a direction, such as a curved or straight direction, having a measurement along the body that is greater than the measurement of the width of the body at that point). Annuloplasty devices, as disclosed herein, may also be symmetrical with respect to one or more planes (e.g., only one plane, or only two planes passing through the device).

The disclosed devices (e.g., devices shaped as a ring) have a cross-sectional profile along a length of the device or at a point along the device. Such a cross-sectional profile may be transverse (e.g., perpendicular or substantially perpendicular) to the longitudinal body of the device at a point along the body of the device. As such, the disclosed devices have a longitudinal central line or axis (e.g., an axis of symmetry and/or a line that is curved) within the ring at the center most point (e.g., in a device having a circular cross sectional profile) or substantially center most point (e.g., in a ring having a non-circular cross sectional profile) of a cross-sectional profile of a device. In some embodiments, a cross-sectional profile of an annuloplasty device may be defined by a plane. As such, the longitudinal central axis of an annuloplasty device may be perpendicular or substantially perpendicular to a plane defining a cross-sectional profile at a point along the device. In various aspects of the device in which a cross-sectional profile of an annuloplasty device is defined by a plane, and the periphery of the device (e.g., one or more outer surfaces of the device) may include one or more curved surfaces and/or one or more straight (i.e., non-curved) surfaces. Embodiments of the described devices include an annuloplasty device in which a cross-sectional profile of the annuloplasty device is defined by a plane, and the periphery of the device (e.g., one or more outer surfaces of the device) in the cross sectional profile is shaped substantially like a capital letter “D” (e.g., have one straight portion and one curved portion and/or wherein the straight portion joins the curved portion at a first and second edge, such as a rounded edge). In some embodiments of an annuloplasty device in which a cross-sectional profile of the annuloplasty device is defined by a plane, and the periphery of the device (e.g., one or more outer surfaces of the device) defines one or more curved surfaces, the plane may include one or more (e.g., two or more) radii of curvature between the periphery of the device and another point (e.g., the longitudinal central line or axis).

In some embodiments of the disclosed devices, a cross-sectional profile of a device may be the same (e.g., have the same cross-sectional area and/or peripheral shape and/or peripheral circumference) at all points along the length of the device. For example, the cross-sectional profile at all points along the length of an annuloplasty device may be shaped as a circle or ovoid having a single area. An annuloplasty device may have the same height and/or thickness along the length of the device or along a portion of the length of the device. In addition, the cross-sectional profile at one or more points along a longitudinal length of an annuloplasty device may be defined by the outer periphery of the annuloplasty device at that point or points and may, in some variations of the disclosed aspects, be shaped as a circle, semi-circle, oval, rectangle, square, triangle, polygon, quadrilateral, or any combination thereof.

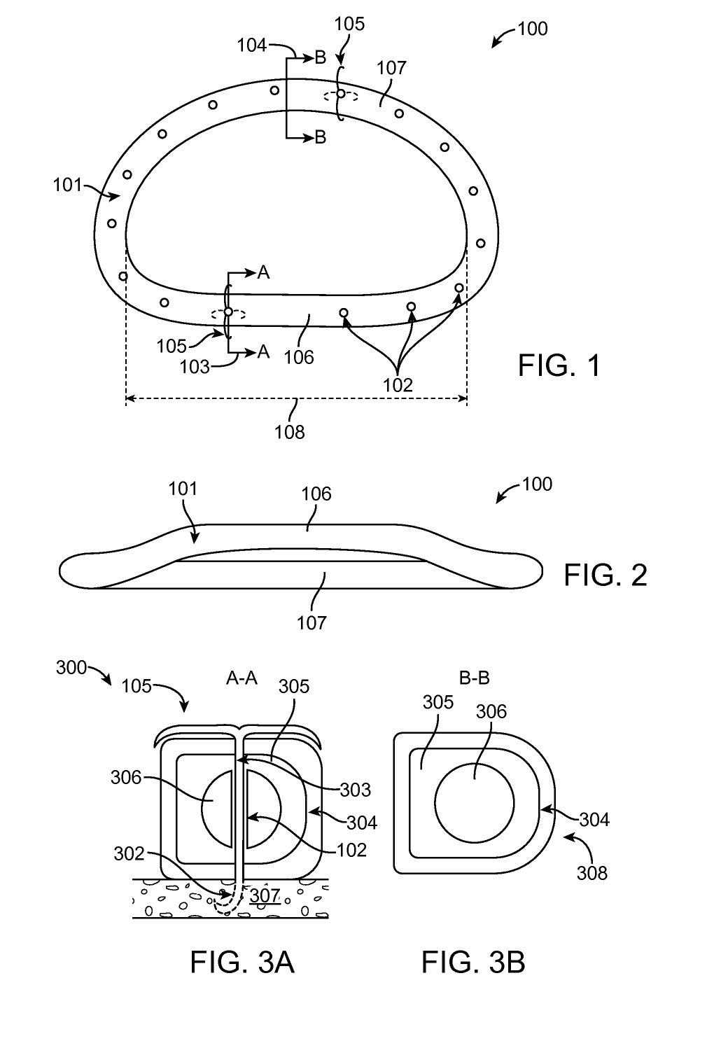

FIG. 1 provides a diagram of a ring shaped annuloplasty device 100 including tissue securing regions 102 according to embodiments of the present disclosure. The diagram of FIG. 1 also shows tissue securing devices 105 according to embodiments of the present disclosure. For illustrative purposes, portions of the tissue securing devices 105 are shown at an angle. Also shown in FIG. 1 are planar 107 and non-planar 106 portions of the device. FIG. 1 illustrates a first cross sectional plane 104 of an annuloplasty device (denoted B-B), wherein the cross section is at a location along the annuloplasty device body 101 so that the cross section does not include a tissue securing region. FIG. 1 also depicts a second cross sectional plane 103 of an annuloplasty device (denoted A-A), wherein the cross section is at a location along the annuloplasty device body 101 so that the cross section includes a tissue securing region. Additionally, FIG. 2 depicts a lateral view of an annuloplasty device 100 and the body thereof 101 according to various embodiments of the present disclosure. Also shown in FIG. 2 are planar 107 and non-planar 106 portions of the device.

As noted above, various aspects of the disclosed annuloplasty devices may include devices that are shaped as a partial ring. For example, annuloplasty devices may be shaped as a ring (e.g., a circular or ovoid ring) having a portion missing from the continuous body of material forming the ring. Annuloplasty devices shaped as a partial ring may be shaped substantially like a capital letter “C”. Annuloplasty devices shaped as a partial ring may include any of the characteristics described herein of annuloplasty devices shaped as a ring but for that the device has the characteristic of being shaped as a complete continuous band or tube of material. In certain aspects of annuloplasty devices shaped as a partial ring, the devices include a body of material having a first end and a second end. Such a body of material may have curved and or straight portions along its length. Such a body of material may also have a first portion (e.g., a first half) that is symmetrical is shape and/or composition of materials to a second portion (e.g., a second half). Examples of annuloplasty devices shaped as a partial ring having characteristics that may be utilized either wholly or partially in connection with the disclosed devices and methods are provided by U.S. Pat. Nos. 8,287,591; 8,382,828; 8,163,012; 8,287,591; 8,123,800; 6,217,610; 8,114,155; 7,879,087; 6,964,684; 6,602,289; 6,416,549; 6,749,630; 6,416,548; 6,908,482; and 6,187,040; as well as published U.S. Pat. App. Publication Nos. 20120221101; 20110022169; 20050256568; 20070276478; 20120330412; and 20120053687; the disclosures of each which are incorporated by reference herein.

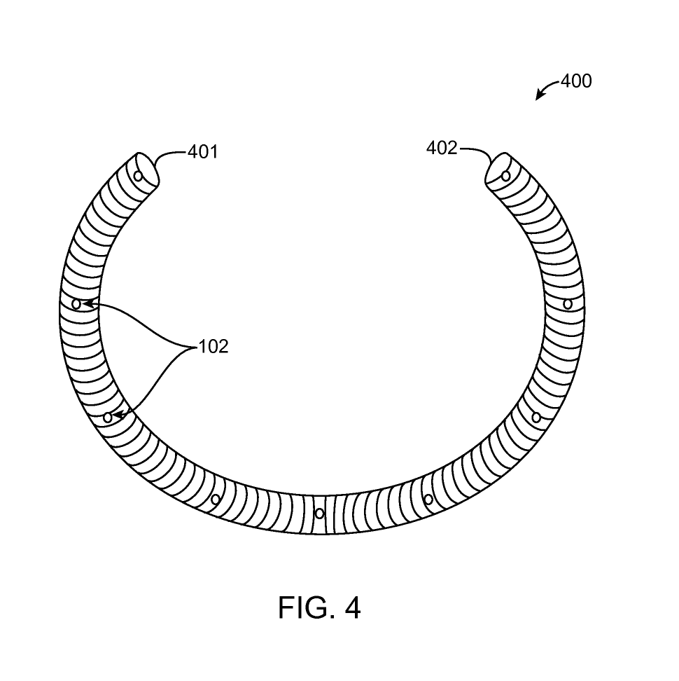

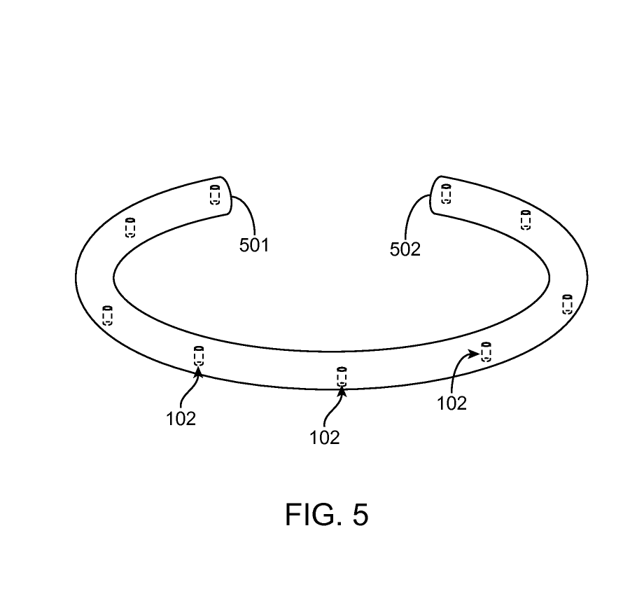

An illustration of an annuloplasty device 400 having a partial ring shape is provided in FIG. 4. FIG. 4 provides a top view of the device and specifically shows a first end 401 and a second end 402 of the device. Also shown in FIG. 4 are tissue securing regions 102 of the device. FIG. 5 provides a perspective view of an annuloplasty device 500 having a partial ring shape in accordance with certain embodiments of the disclosed devices. A first end 501 and second end 502 of the device are also depicted in FIG. 5. Also shown in FIG. 5 are tissue securing regions 102 of the device.

Variations of the disclosed devices also include annuloplasty devices shaped as a saddle. For example, devices shaped as a saddle may be bent down at the sides (e.g., first and second opposing sides) so as to give an upper part (e.g., first and/or second rounded regions) a rounded form. Annuloplasty devices shaped as a saddle are non-planar and/or may be shaped as a full or partial ring. For example, in an annuloplasty device that is saddle-shaped, all of the upper-most points on the top surface of an annuloplasty device are not in a first plane and/or all of the lower-most points on the bottom surface of an annuloplasty device are not in a second plane. Examples of annuloplasty devices shaped as a saddle and having characteristics that may be utilized either wholly or partially in connection with the disclosed devices and methods are provided by U.S. Pat. Nos. 8,236,050; 7,452,376; 8,382,828; 6,805,710; 6,908,482; 6,749,630; 5,888,240; 5,593,435; 8,114,155; 8,163,012; and 7,993,395; as well as published U.S. Pat. App. Publication Nos. 20120053687; 20060206203; 20070100441; 20080058924; 20050256568; 20090036979; 20030208264; 20010021874; 20050131533; 20060100697; 20120215304; 20130030523; 20110093065; the disclosures of each which are incorporated by reference herein.

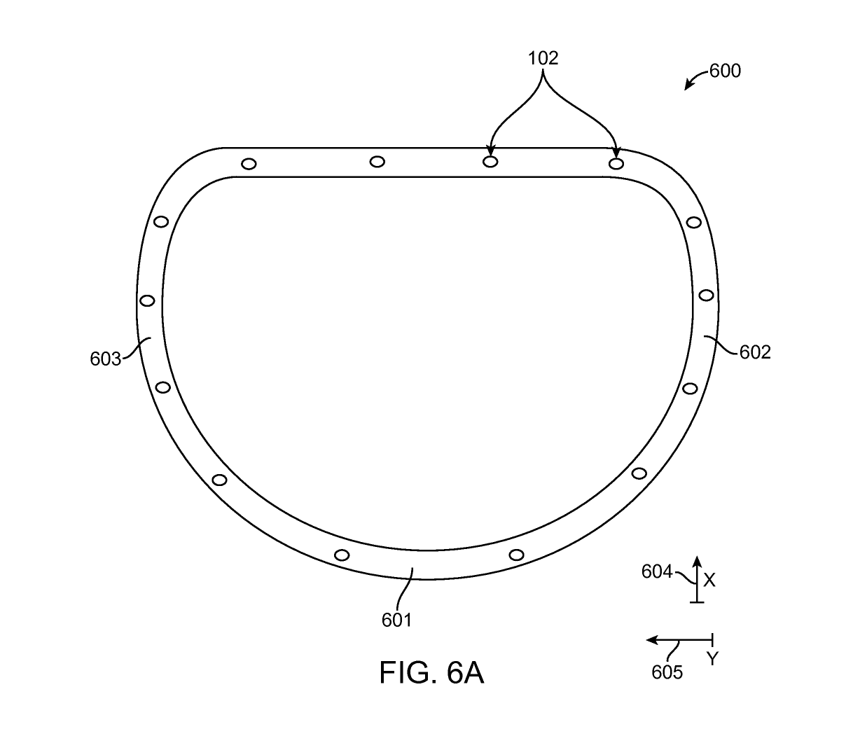

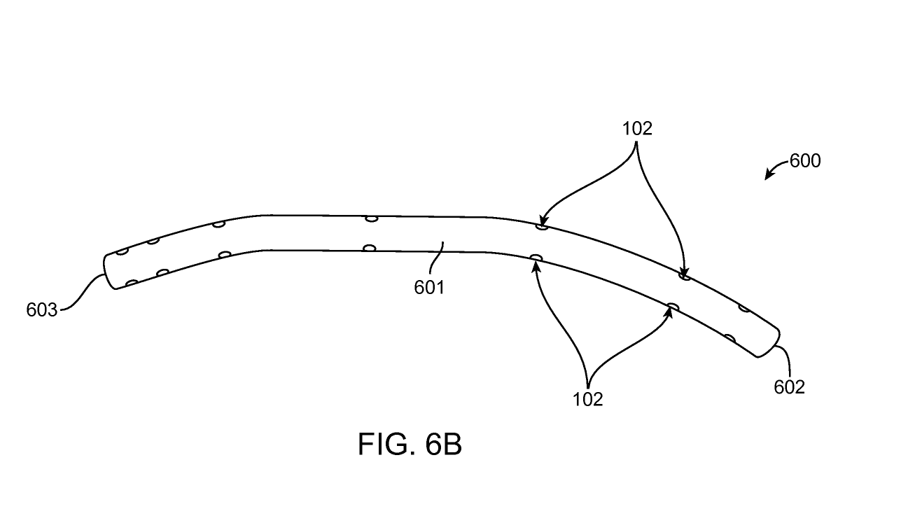

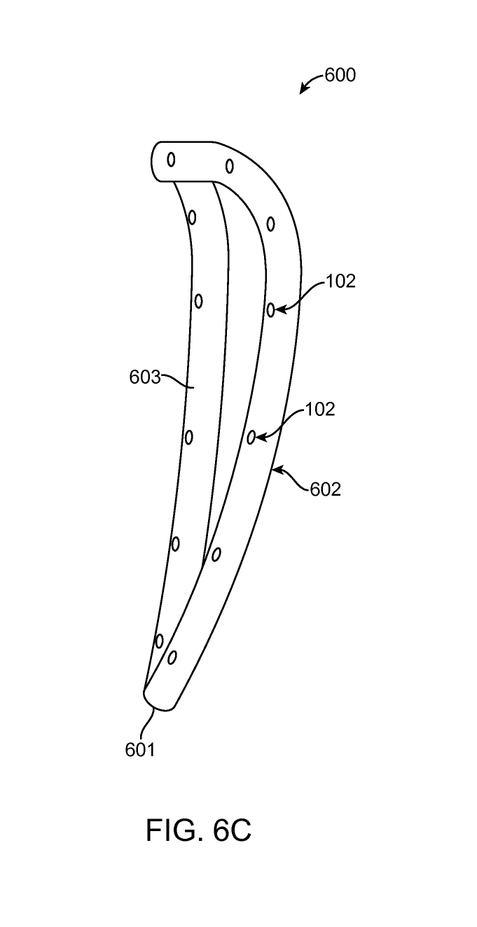

A depiction of a saddle shaped annuloplasty device 600 is provided in FIGS. 6A-C. Specifically shown in FIG. 6A are reference portions A 601, B 602, and C 603 of the annuloplasty device body. The annuloplasty device shown also includes tissue securing regions 102. Also provided in FIG. 6A are reference directions X 604 and Y 605. A lateral view of the saddle shaped annuloplasty device 600 taken in the direction X 604 is provided in FIG. 6B. FIG. 6B illustrates tissue securing regions in the body of the annuloplasty device as well as reference portions A 601, B 602, and C 603. A view of the saddle shaped annuloplasty device 600 taken in the direction Y 604 is provided in FIG. 6C. FIG. 6C specifically illustrates reference portions A 601, B 602, and C 603 of annuloplasty device body. Also shown in FIG. 6C are tissue securing regions 102 of the device.

Embodiments of annuloplasty rings of the subject devices are also configured to be implantable. As noted above, a longitudinal central axis of an annuloplasty device (e.g., an implantable annuloplasty device) may be perpendicular or substantially perpendicular to a plane defining a cross-sectional profile at a point along the device. As such, the thickness of an annuloplasty device may be defined by the distance from one point on the outer periphery of the cross section of the device to another on the opposite side of the periphery in a horizontal direction. Such a thickness of an annuloplasty device (e.g., an implantable annuloplasty device) may range from, for example, 0.3 mm to 10 mm, such as from 1 mm to 5 mm or 1 mm to 3 mm. Similarly, the height of an annuloplasty device may be defined by the distance from one point on the outer periphery of the cross section of the device to another on the opposite side of the periphery in a vertical direction. Such a height of an annuloplasty device may be greater then, less than or equal to the thickness of the device and range from, for example, 0.3 mm to 10 mm, such as from 1 mm to 5 mm or 1 mm to 3 mm.

In some embodiments of the disclosed annuloplasty devices, the devices are planar or substantially planar. For example, in annuloplasty devices that are planar, an annuloplasty device or portion thereof (e.g., the one or more exterior surfaces of the device) may all fit within a plane having a thickness corresponding with the thickness (e.g., the distance from one point on the outer periphery of the cross section of the device to another on the opposite side of the periphery) of the annuloplasty device.

Additionally, in some embodiments, annuloplasty devices have a top surface (e.g., a surface oriented upward when the device is placed on a surface and/or implanted into the body of a subject) and a bottom surface (e.g., a surface oriented downward when the device is placed on a surface and/or implanted into the body of a subject). In an annuloplasty device that is planar, all of the upper-most points on the top surface of an annuloplasty device are in a first plane and/or all of the lower-most points on the bottom surface of an annuloplasty device are in a second plane. Some embodiments of annuloplasty devices (e.g., annuloplasty devices that are substantially planar) are shaped so that the majority (i.e., more than half) of the upper-most points on the top surface of the annuloplasty device are in a first plane and/or the majority (i.e., more than half) of the lower-most points on the bottom surface of the annuloplasty device are in a second plane.

Furthermore, an implantable annuloplasty device (e.g., an annuloplasty device having a ring or partial ring shape) may have an inner length or diameter dimension, which is the length (e.g., diameter) measured on a device resting on a surface, in a horizontal direction from one inner-most point on the inner periphery of a ring to a similar point on the opposite side of the ring (e.g., from such opposing points located at the furthest possible distance on a device). Such a measurement is shown in FIG. 1 as length 108. An inner length (e.g., diameter) of an annuloplasty device may range for example, from 5 mm to 50 mm, such as from 10 mm to 40 mm, including 20 mm to 40 mm. Similarly, the circumference or partial circumference (e.g., distance from one end to an opposite end along the longitudinal body of the device) of an annuloplasty device may range from 5 mm to 160 mm, such as from 30 mm to 145 mm, including from 60 mm to 125 mm.

Embodiments of annuloplasty devices and/or the materials thereof may have one or more flexibilities and/or rigidities. The term “flexible” is used in its conventional sense to mean capable of being bent, usually without breaking, e.g., easily bent. The term “rigid” is also used in its conventional sense to mean stiff or unyielding, e.g., not pliant or flexible, in other words hard. In certain aspects, an annuloplasty device or one or more portions or materials thereof is flexible (e.g., the device can be flexed when pressure is applied to the device by a human heart or hand). In certain aspects, an annuloplasty device or one or more portions or materials thereof is rigid (e.g., the device cannot be easily flexed or flexed at all when pressure is applied to the device by a human heart or hand). In certain aspects, an annuloplasty device or one or more portions or materials thereof is semi-rigid. In some embodiments, an annuloplasty device includes at least two (e.g., two, three, four, five, six, seven, eight, etc.) rigid regions (e.g., a region composed of one or more rigid materials) and/or at least two semi-rigid regions (e.g., a region composed of one or more semi-rigid materials) and/or at least two flexible regions (e.g., a region composed of one or more flexible materials).

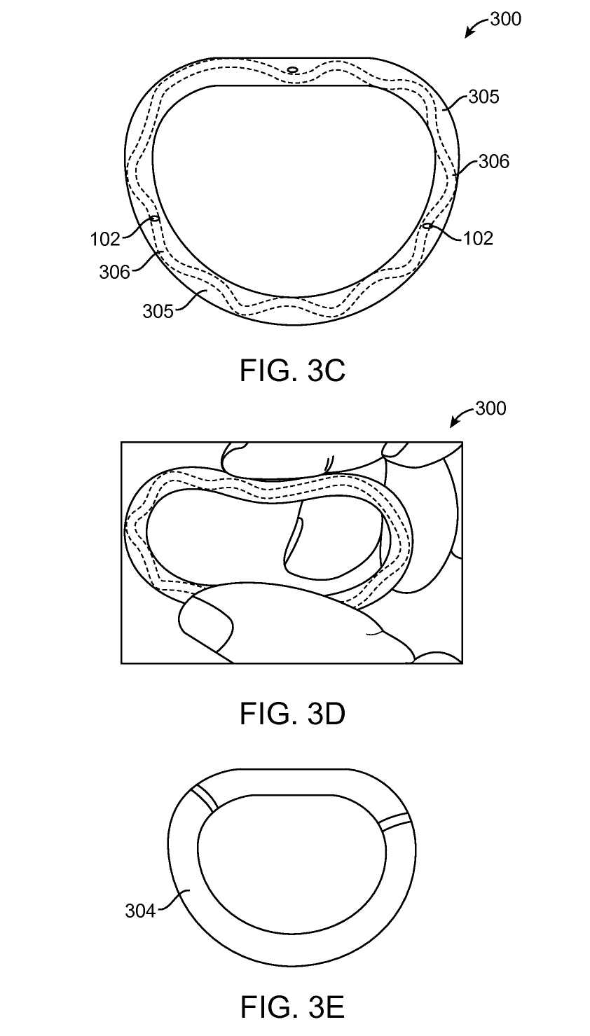

The disclosed devices may be composed of a wide variety of one or more materials. In some embodiments of the described annuloplasty devices, the body of the device is a partial ring or ring including or composed of a first material (e.g., the “first” material) (e.g., shape memory materials, such as a metal alloy, e.g., as ELGILOY® nickel cobalt chromium alloy or NITINOL® alloy) and a tissue securing region of the device includes a void (e.g., a void in the body of the device) and/or a portion which may be completely or partially filled with a second material (e.g., the “second” material) (e.g., silicone) that is more compliant (e.g., more malleable, softer, less rigid, less dense, and/or more readily pierced by an object such as a tissue securing device) than the first material. For example, the device may include a ring of a shape memory material coated with the second material, e.g., a silicone, where tissue securing regions are present in the first shape memory material. In some embodiments, the devices include on or more components (e.g., securing members) made of a shape memory material. Shape memory materials are materials that exhibit the shape memory effect, where the materials that have a temperature induced phase change, e.g., a material that if deformed when cool, returns to its “undeformed”, or original, shape when warmed, e.g., to body temperature. Where desired, the shape memory material may be one with a transformation temperature suitable for use with a stopped heart condition where cold cardioplegia has been injected for temporary paralysis of the heart tissue (e.g., temperatures as low as 8-10 degrees Celsius). The shape memory material may also be heat activated, or a combination of heat activation and pseudoelastic properties may be used. Shape memory materials of interest include shape memory metal alloys, such as alloys of nickel (e.g., nickel titanium alloy (NITINOL®), nickel cobalt alloys (e.g., ELGILOY® cobalt-chromium-nickel alloy, etc.), zinc, copper (e.g., CuZnAI), gold, iron, etc. Also of interest are non-metallic materials that exhibit shaper memory qualities, e.g., shape memory plastics, etc. In embodiments where a ring of shape memory material is encased in a second material, such as a silicone or analogous material (e.g., polymeric material), the ring of shape memory material may have a variety of configurations, and in some instances may have an undulating configuration, e.g., as illustrated in FIGS. 3C and 3D.

Disclosed devices may also include a coating component which coats (e.g., fully or partially encapsulates or contains) at least a portion of the annuloplasty device body. In such embodiments, at least a portion of an annuloplasty device body is between two portions of the coating component. In some embodiments, the coating component includes a material (e.g., the “second” material) that is more compliant than a material of which at least a portion of the annuloplasty device body is composed (e.g., the “first” material). In some embodiments, the “second” material includes silicone (e.g., an amount of silicone having the same volume as one or more tissue securing regions of the device). In some embodiments, the “second” material is applied to one or more other materials of the annuloplasty devices by dipping (e.g., dipping one or more materials of an annuloplasty device, such as ELGILOY® nickel cobalt chromium alloy, into another material, such as silicone).

In some embodiments, annuloplasty devices may include an outer cover composed of a material (e.g., a “third” material) that may be different than a material (e.g., the “second” material) that is more compliant than the material of which at least a portion of the annuloplasty device body is composed (e.g., the “first” material). The outer cover of devices may, in some versions, fully or partially encapsulate at least a portion (e.g., the entirety of) of the annuloplasty device body and/or tissue securing regions. In some embodiments, one or more materials of which an outer cover of a device is composed (e.g., the “third” material) includes one or more fabrics (e.g., a fabric including polyester fibers). In some versions, one or more materials of which an outer cover of a device is composed include Dacron™.

In various embodiments of the disclosed devices, one or more materials of the devices are arranged in one or more (e.g., one, two, three, four or five) concentric layers. One or more concentric layers of material in such a device may encapsulate or substantially encapsulate one or more other layers.

Devices as described herein may be fabricated from any convenient material or combination of materials. Materials of interest include, but are not limited to: polymeric materials, e.g., plastics, such as polytetrafluoroethene or polytetrafluoroethylene (PFTE), including expanded polytetrafluoroethylene (e-PFTE), polyester (Dacron™), nylon, polypropylene, polyethylene, high-density polyethylene (HDPE), polyurethane, etc., metals and metal alloys, e.g., titanium, chromium, stainless steel, etc., and the like. In some embodiments, the devices include on or more components made of a shape memory material. Shape memory materials are materials that exhibit the shape memory effect, where the materials that have a temperature induced phase change, e.g., a material that if deformed when cool, returns to its “undeformed”, or original, shape when warmed, e.g., to body temperature. Where desired, the shape memory may be one with a transformation temperature suitable for use with a stopped heart condition where cold cardioplegia has been injected for temporary paralysis of the heart tissue (e.g., temperatures as low as 8-10 degrees Celsius). The shape memory material may also be heat activated, or a combination of heat activation and pseudoelastic properties may be used. Shape memory materials of interest include shape memory metal alloys, such as alloys of nickel (e.g., nickel titanium alloy (NITINOL®), nickel cobalt alloys (e.g., ELGILOY® nickel cobalt chromium alloy, etc.), zinc, copper (e.g., CuZnAl), gold, iron, etc. Also of interest are non-metallic materials that exhibit shaper memory qualities, e.g., shape memory plastics, etc.

FIG. 3A provides a diagram depicting a cross-sectional view of an annuloplasty device 300 taken at plane 103 and including a tissue securing region 102 according to embodiments of the present disclosure (line A-A as depicted in FIG. 1). The annuloplasty device 300 shown in FIG. 3Q includes an outer coverling I portion (e.g., layer) 304 of a suitable material (e.g., a layer including Dacron™), a second portion 305 (e.g., a layer including silicon, such as silicon applied to the device by dipping), and a third portion 306 (e.g., a central core of a shape memory material, such as NITINOL® alloy or ELGILOY® nickel cobalt chromium alloy and/or other shape memory material). The diagram of FIG. 3A also shows a tissue securing device 105 according to embodiments of the present disclosure. The tissue securing device 105 includes a second deployable arm 302 and has a body portion 303. FIG. 3A also depicts tissue 307 (e.g., a tissue layer).

FIG. 3B provides a diagram showing a cross-sectional view of an annuloplasty device 308 taken at plane 104 and not including a tissue securing region according to embodiments of the present disclosure (line B-B as depicted in FIG. 1). The annuloplasty device 300 shown in FIG. 3B includes a first portion (e.g., outer layer) 304 (e.g., a layer including Dacron™), a second portion 305 (e.g., a layer including silicon, such as silicon applied to the core of the device by dipping), and a third portion 306 (e.g., a central core of the shape memory material, such as NITINOL® alloy ELGILOY® nickel cobalt chromium alloy and/or other shape memory material). FIGS. 3C to 3D provide additional views of the device depicted in FIGS. 3A and 3B.

### Tissue Securing Devices

As noted above, embodiments of the disclosed annuloplasty devices include devices having a body and one or more tissue securing regions. Tissue securing regions are portions of annuloplasty devices which may be configured (e.g., sized and/or shaped) to fully and/or partially receive and/or retain one or more tissue securing devices therein.

Embodiments of tissue securing devices include implantable devices configured to attach annuloplasty devices to tissue of a subject. In certain aspects, tissue securing devices or portions thereof are configured to be introduced (e.g., inserted) through tissue securing regions of annuloplasty devices and into tissue of a subject to thereby anchor the annuloplasty devices to the tissue.

In certain embodiments, tissue securing devices, which may include an implantable body, have a first portion (e.g., the “first” portion) (e.g., a distal deployable arm) which is introducible (e.g., insertable) into and through tissue securing regions and is introducible into tissue of a subject. The first portion of a tissue securing device may include a first (e.g., distal) end of the device. Tissue securing devices and/or the implantable body thereof may also have a second portion (e.g., the “second” portion), of which at least a portion is introducible into and retainable within tissue securing regions. In addition, tissue securing devices and/or the implantable body thereof may also have a third portion (e.g., the “third” portion) (e.g., a proximal deployable arm), of which at least a portion is configured to contact and thereby retain annuloplasty devices against adjacent tissue. The third portion of a tissue securing device may include a second (e.g., proximal) end of the device.

In some embodiments of tissue securing devices, the “first” portion includes one or more tissue engagers, such as hooks and/or barbs configured to engage tissue of a subject. The “first” portion of tissue securing devices may have one or more portions that are curved and/or straight. In various embodiments, the “second” portion is a rod (e.g., a circular rod) of one or more materials connecting the “first” portion and the “third” portion. In some versions, the “third” portion includes one or more tissue engagers, such as hooks and/or barbs configured to abut and be retained against at least a portion of an annuloplasty device. The “third” portion of tissue securing devices may have one or more portions that are curved and/or straight.

Embodiments of tissue securing devices include a body (e.g., an implantable body and/or a longitudinal body) having a first end and a second end and the first end and/or the second end is barbed (e.g., includes one or more barbs thereon). In some embodiments, barbs may include a body of one or more materials that is shaped to engage (e.g., interlock with, such as in a non-releasable manner) tissue. Certain embodiments of barbs engage tissue by piercing tissue and/or traveling through tissue and/or being arranged within tissue in a manner that exerts force on the tissue such that the barb or the aspect to which the barb is attached remains within and/or attached to the tissue. In various aspects, barbs may include a point (e.g., a sharp point) projecting in a reverse direction (e.g., at an angle, such as an obtuse angle, away from) than an end and/or main point of a device or aspect.

In some embodiments, the “first” and “third” portions of tissue securing devices have the same shape. Embodiments of tissue securing devices also include those in which the “first” and “third” portions of tissue securing devices each are in the same plane (e.g., the plane having the thickness of the device) or in different (e.g., perpendicular and/or orthogonal) planes (e.g., the planes each respectively having the thickness of the portion of the device within the plane).

In some aspects of the disclosed devices, the “first” and “third” portions of tissue securing devices and/or the proximal and/or distal deployable arms have one or more different configurations (e.g., a first and a second configuration, such as a curved and/or deployed configuration and a straight and/or non-deployed configuration) and each portion may be biased to be retained in one of the different configurations (e.g., a curved and/or deployed configuration).

As noted above, in certain embodiments of tissue securing devices, the devices include an implantable body (e.g., a portion of one or more materials, such as any of the materials described herein) having a proximal deployable arm at a proximal end and/or a distal deployable arm at a distal end. In such embodiments, the proximal and distal deployable arms may be in a non-coplanar (e.g., perpendicular and/or orthogonal) configuration. In various embodiments of the devices, the proximal and distal deployable arms each define a plane which has the thickness of each respective corresponding arm and which are perpendicular to one another.

The disclosed devices include tissue securing devices which are composed of one or more materials, such as the materials described herein (e.g., the shape memory materials described herein). The disclosed devices also include tissue securing devices or one or more portions thereof (e.g., the “first”, “second”, or “third” portions) that are configured (e.g., sized and/or shaped) to be introducible (e.g., insertable) into or through and/or be retained within the tissue securing regions described herein. In some embodiments, tissue securing devices may have a dimension, (e.g., cross-sectional diameter, length, such as a longitudinal length, width or height), such as a longest dimension, ranging from 0.1 mm to 10 mm, such as from 1 mm to 5 mm, including from 1 mm to 2 mm.

As noted above, FIG. 1 shows tissue securing devices 105 according to embodiments of the present disclosure. The diagram of FIG. 3a also shows a tissue securing device 105 within a tissue securing region 102 of an annuloplasty device according to embodiments of the present disclosure. The tissue securing device 105 includes a first deployable arm 301, a second deployable arm 302 and has a body portion 303.

Further examples of tissue securing devices or characteristics thereof that may be utilized, either wholly or partially, in connection with the disclosed annuloplasty devices and methods are provided by U.S. Pat. Nos. 6,447,524; 6,425,900; 5,582,611; 7,150,750; 6,113,611; 6,290,702; 6,325,805; 4,627,437; 7,056,330; 8,292,154; 6,074,418; 7,828,187; 8,282,670; 4,261,244; 4,887,601; 4,489,875; 5,366,479; 7,056,333; 5,456,400; 5,964,772; 389,660; and 5,342,376, as well as U.S. Pat. App. No. 61/831,454 and international application serial no. PCT/US2014/______ having Attorney Docket No. LCTHX-008WO and titled “TISSUE ANCHOR AND DEPLOYMENT DEVICE FOR SAME”, filed on Jun. 4, 2014; as well as U.S. Published Application Nos. 20030097148; 20030229360; and 20020117534; the disclosures of each which are incorporated by reference herein.

### Methods

The subject devices find use in methods for fastening (i.e., stably associating) an annuloplasty device as described herein (e.g., an annuloplasty ring) to a tissue, such as a cardiac valve annulus (e.g., mitral valve annulus) with a tissue securing device. By “stably associating” is meant that the device is substantially if not completely fixed relative to the tissue location of interest. Methods implanting an annuloplasty device for repair of a cardiac valve, such as a mitral valve, are discussed below. When performing an annuloplasty ring implantation procedure, incisions may be made into the thoracic cavity and pericardium, and then into aorta or myocardium in order to have access to the damaged heart valve tissue site. The procedure may be an open procedure in which the sternum is opened and the ribs are spread with a conventional retractor, or a minimally invasive procedure, e.g., wherein the heart and heart valve are accessed through minimally invasive openings in the thoracic cavity, such as through trocar cannulas or small incisions in the intercostal spaces, via blood vessels, etc. The minimally invasive procedures can be viewed remotely using a camera and monitor, or in some cases directly, as desired.

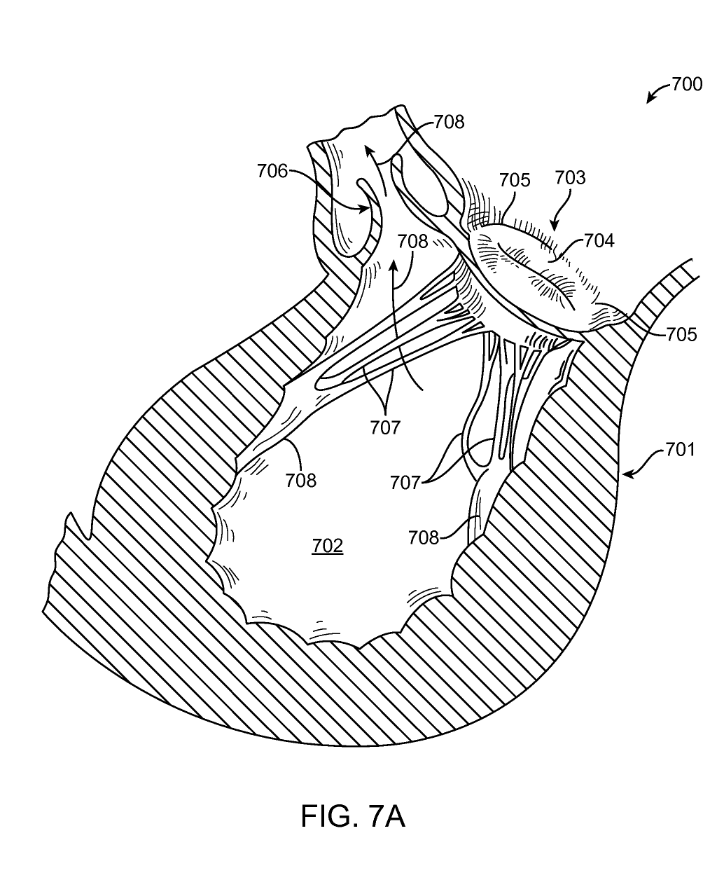

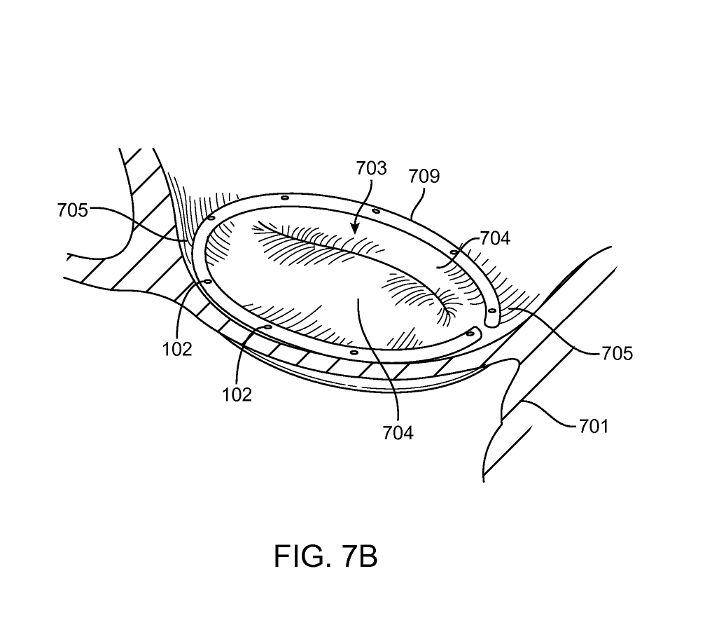

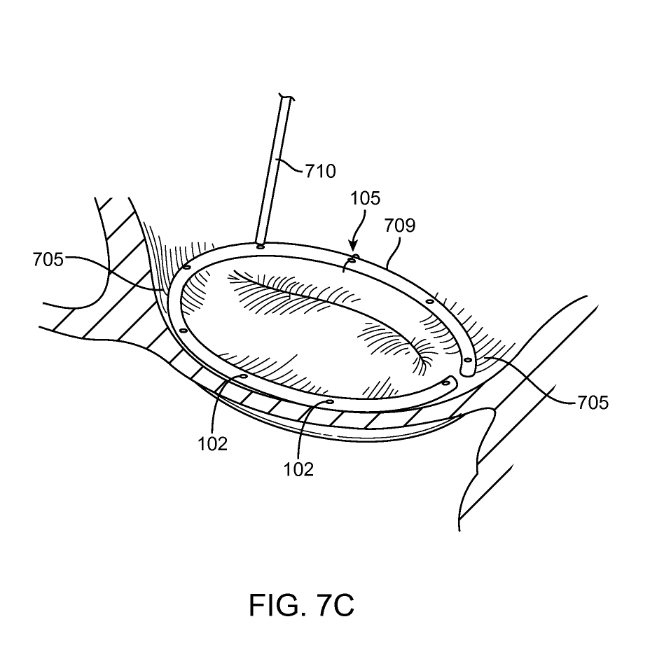

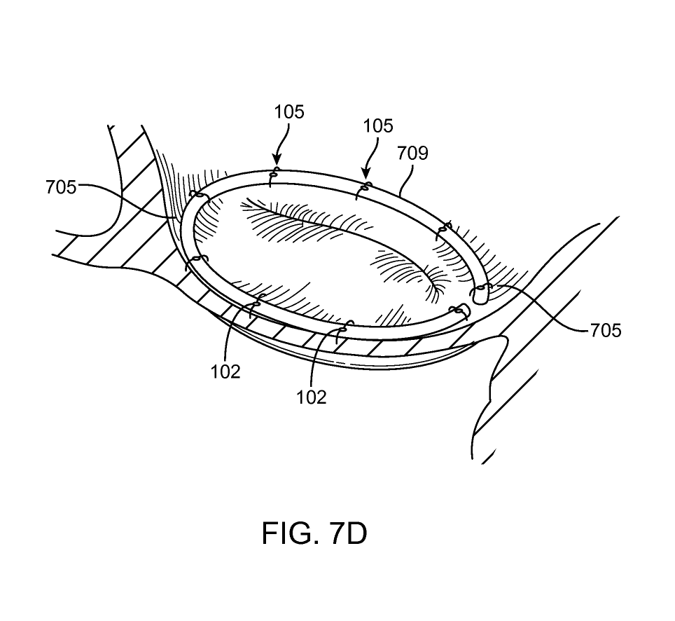

FIG. 7A illustrates the step of exposing a cardiac valve for implantation 700 of an annuloplasty device. Provided in FIG. 7A is a cross sectional view of a left ventricle 702 of a heart 701. More specifically, FIG. 7A shows an exposed mitral valve 703 having leaflets 704 and annulus 705. Also depicted in FIG. 7A are the aortic valve 706 and chordae tendinae 707 extending between papillary muscles 708 and the mitral valve leaflets 704. The direction of blood flow out of the left ventricle is shown by arrows 708.

After the cardiac valve (e.g., the mitral valve) is exposed, the desired size and/or shape of the annuloplasty device is determined by measuring the distance between the anterior and posterior comissures, the anterior leaflet height and/or the surface area of the anterior and posterior mitral leaflets. The desired size and/or shape for the annuloplasty device can be determined using any suitable measuring device, such as a caliper. The measurement can also be confirmed by comparison with pre-operative transesophageal echocardiography (TEE).

An annuloplasty device having a desired size and shape, or the closest to the desired size and shape, is then selected from among a set of annuloplasty devices. The set of annuloplasty devices can include two or more annuloplasty devices of the same or of different sizes and/or shapes, such as three devices, or four devices, etc. The annuloplasty device is then advanced into the heart, such as into the left atrium, in some instances by using a delivery device, and seated onto a valve annulus (e.g., the mitral valve annulus).

In certain embodiments, the methods include steps (e.g., sequential steps and/or simultaneous steps) of (1) positioning an annuloplasty device at a tissue site (e.g., a cardiac valve site) and (2) engaging tissue of the tissue site with a tissue securing device in a manner sufficient to stably implant the annuloplasty device at the tissue site.

As used herein, the phrases “stably implant” and “stably implanting” refer to implanting one or more objects (e.g., an annuloplasty device) into the body of a subject and affixing the one or more objects to tissue therein in a secure manner (e.g., in a manner such that the one or more objects is retained at the same position or substantially at the same position within the body of a subject for a time period, such as a for a period of months, years and/or for the remaining lifetime of the subject or more).

As noted above, the methods disclosed herein also include implanting an annuloplasty device at a tissue site (e.g., a cardiac valve site, such as a mitral valve site). As used herein, the phrase “tissue site” refers to a location within the body of a subject at, contacting, within, above, below and/or immediately adjacent to (e.g., within 1, 2, 3, 4, 5, 6, 7, 8, 9, or 10 mm of) one or more tissues (e.g., the annulus of a cardiac valve) to which a device (e.g., an annuloplasty device) may be attached. In some embodiments, a “tissue site” is a cardiac valve site (e.g., a mitral valve site). As used herein, the phrase “cardiac valve site” refers to the location within the body of a subject at, contacting, within, above, below and/or immediately adjacent to (e.g., within 1, 2, 3, 4, 5, 6, 7, 8, 9, or 10 mm of) one or more cardiac valves (e.g., the mitral, tricuspid, aortic and/or pulmonary valves). Accordingly, the phrase “mitral valve site” refers to the location within the body of a subject at, contacting, within, above, below and/or immediately adjacent to the mitral valve and/or a portion (e.g., annulus) thereof.

The methods disclosed herein may also include positioning a device (e.g., an annuloplasty device) at a tissue site, such as onto a cardiac valve annulus. The step of positioning an annuloplasty device 705 having tissue securing regions 102 at a tissue site is shown in FIG. 7B. FIG. 7B specifically illustrates an annuloplasty device 709 seated on annulus 705 at the mitral valve 703 of a heart 701. FIG. 7B also illustrates the position of the annuloplasty device 709 with respect to the leaflets 704 of the mitral valve. While positioned at the tissue site, the annuloplasty device 709 may be held in the proper position by one or more instruments (not shown).

As is shown in FIG. 7C, once the annuloplasty device 709 is in the proper position, it may be retained in that position while a tissue securing region 102 on the device is identified and an apparatus 710 for deploying a tissue securing device is placed in an orientation (e.g., proximity and/or angle) to deploy a tissue securing device into the tissue securing region 102 of the annuloplasty device 709. The apparatus 710 for deploying a tissue securing device may then be actuated to deploy a portion of a tissue securing device 105 through a tissue securing region 102 to engage the tissue of the tissue site below (e.g., the annulus 705) or adjacent to the annuloplasty device 709. A deployed tissue securing device 105 is shown in FIG. 7C.

As such, embodiments of the disclosed methods also include engaging tissue of the tissue site with a tissue securing device. The word “engaging” is used herein to refer to contacting a first aspect (e.g., a tissue) with a second aspect (e.g., a tissue securing device) and/or affixing and/or anchoring the first aspect to the second aspect. “Engaging” may refer to piercing a first aspect (e.g., a tissue) with a second aspect (e.g., a tissue securing device) and causing the second aspect, or a portion thereof, to contact the first aspect in a non-releasable manner, such as by deploying one or more tissue engagers, such as hooks and/or barbs within the first aspect. When a portion of a tissue securing device is deployed through a tissue securing region to engage the tissue of the site (e.g., annulus) below, a portion of the tissue securing device may be retained on the upper surface of the annuloplasty device to hold the annuloplasty device against the underlying annulus.

In various aspects, the disclosed methods may include engaging tissue of the tissue site with one or more tissue securing devices. FIG. 7D illustrates an annuloplasty device 709 that has been affixed to the underlying mitral valve annulus 705 (e.g., stably implanted) by tissue securing devices 105 deployed through each of the tissue securing regions 102 of the device.

The number of such tissue securing devices used in accordance with the disclosed methods (e.g., in implanting an annuloplasty device) may be any of the numbers listed above in describing how many tissue securing regions that embodiments of annuloplasty devices may have. In methods which include engaging tissue of the tissue site with two or more tissue securing devices, a deploying apparatus for deploying a tissue securing device is re-oriented after deploying a first tissue securing device to deploy a tissue securing device into a second tissue securing region of the annuloplasty device and actuated to deploy a portion of a tissue securing device through the second tissue securing region to engage tissue of the tissue site below or adjacent to the annuloplasty device. The process of re-orienting the deploying apparatus and deploying a portion of a tissue securing device through a tissue securing region to engage underlying and/or adjacent tissue is repeated until the annuloplasty device is securely affixed to the tissue site (e.g., annulus).

Embodiments of the disclosed methods include engaging tissue of the tissue site with a tissue securing device that is operably associated with a tissue securing region. As used herein, the phrase “operably associated” means associated with or causing to be associated with in a specific way (e.g., stably implanted) (e.g., in a manner in which one aspect, such as a tissue securing device, is introduced (e.g., inserted) at least partially into and/or retained at least partially within another aspect, such as a tissue securing region) that allows the disclosed devices to operate and/or methods to be carried out effectively in the manner described herein.

In certain embodiments, the disclosed methods include methods wherein a tissue securing device is operably associated with a tissue securing region, such as a tissue securing region including a void (e.g., a void that is associated with and/or within the body of an annuloplasty device), by partially passing the tissue securing device through the void (i.e., introducing and causing a portion of the device to travel through the void).

In some versions of the disclosed methods, the method is a surgical procedure. As used herein, the phrase “surgical procedure” refers to a procedure (e.g., a medical procedure) involving at least one incision in the body of a subject and/or performed using one or more instruments (e.g., surgical instruments). A surgical procedure may be carried out through a body cavity and/or through the skin of a subject.

As noted above, in certain variations of the disclosed methods, the method is an open surgical procedure. As used herein, the phrase “open surgical procedure” refers to a surgical procedure wherein at least one long incision (e.g., having a length of 10 cm) is made in the body of a subject to introduce at least one surgical instrument and/or visualize the surgery through the incision. In an open surgical procedure, closure devices, e.g., staples, sutures, etc., may be used to close at least one incision.

In certain variations of the disclosed methods, the method is a minimally invasive surgical procedure. As used herein, the phrase “minimally invasive surgical procedure” refers to a surgical procedure that is less invasive than an open surgical procedure. A minimally invasive surgical procedure may involve the use of arthroscopic and/or laparoscopic devices and/or remote-control manipulation of surgical instruments. Minimally invasive surgical procedures include endovascular procedures, which may be totally endovascular procedures, percutaneous endovascular procedures, etc. Endovascular procedures are procedures in which at least a portion of the procedure is carried out using vascular access, e.g., arterial access.

The subject methods also may include the step of diagnosing a patient in need of cardiac valve repair, e.g., mitral valve repair. Primary mitral regurgitation is due to any disease process that affects the mitral valve device itself. The causes of primary mitral regurgitation include myxomatous degeneration of the mitral valve, infective endocarditis, collagen vascular diseases (e.g., SLE, Marfan's syndrome), rheumatic heart disease, ischemic heart disease/coronary artery disease, trauma, balloon valvulotomy of the mitral valve, and certain drugs (e.g., fenfluramine). If valve leaflets are prevented from fully coapting (i.e., closing) when the valve is closed, the valve leaflets will prolapse into the left atrium, which allows blood to flow from the left ventricle back into the left atrium, thereby causing mitral regurgitation.

The signs and symptoms associated with mitral regurgitation can include symptoms of decompensated congestive heart failure (e.g., shortness of breath, pulmonary edema, orthopnea, and/or paroxysmal nocturnal dyspnea), as well as symptoms of low cardiac output (e.g., decreased exercise tolerance). Cardiovascular collapse with shock (i.e., cardiogenic shock) may be seen in individuals with acute mitral regurgitation due to papillary muscle rupture or rupture of a chorda tendinea. Individuals with chronic compensated mitral regurgitation may be asymptomatic, with a normal exercise tolerance and no evidence of heart failure. These individuals however may be sensitive to small shifts in their intravascular volume status, and are prone to develop volume overload (e.g., congestive heart failure).

Findings on clinical examination depend of the severity and duration of mitral regurgitation. The mitral component of the first heart sound is usually soft and is followed by a pansystolic murmur which is high pitched and may radiate to the axilla. Patients may also have a third heart sound. Patients with mitral valve prolapse often have a mid-to-late systolic click and a late systolic murmur.

Diagnostic tests include an electrocardiogram (EKG), which may show evidence of left atrial enlargement and left ventricular hypertrophy. Atrial fibrillation may also be noted on the EKG in individuals with chronic mitral regurgitation. The quantification of mitral regurgitation usually employs imaging studies such as echocardiography or magnetic resonance angiography of the heart. The chest x-ray in patients with chronic mitral regurgitation is characterized by enlargement of the left atrium and the left ventricle. The pulmonary vascular markings are typically normal, since pulmonary venous pressures are usually not significantly elevated. An echocardiogram, or ultrasound, is commonly used to confirm the diagnosis of mitral regurgitation. Color doppler flow on the transthoracic echocardiogram (TTE) will reveal a jet of blood flowing from the left ventricle into the left atrium during ventricular systole. Because of the difficulty in getting accurate images of the left atrium and the pulmonary veins on the transthoracic echocardiogram, a transesophageal echocardiogram may be necessary to determine the severity of the mitral regurgitation in some cases. The severity of mitral regurgitation can be quantified by the percentage of the left ventricular stroke volume that regurgitates into the left atrium (the regurgitant fraction). Other methods that can be used to assess the regurgitant fraction in mitral regurgitation include cardiac catheterization, fast CT scan, and cardiac MRI.

Indications for surgery for chronic mitral regurgitation include signs of left ventricular dysfunction. These include an ejection fraction of less than 60 percent and a left ventricular end systolic dimension (LVESD) of greater than 45 mm.

Additionally, and as noted above, supplementary examples of tissue securing devices or characteristics thereof that may be utilized either wholly or partially in connection with the disclosed methods are provided by U.S. Pat. App. No. 61/831,454 and international application serial no. PCT/US2014/______ having Attorney Docket No. LCTHX-008WO and titled “TISSUE ANCHOR AND DEPLOYMENT DEVICE FOR SAME”, filed on Jun. 4, 2014; and U.S. Pat. No. 6,447,524; the disclosures of each which are incorporated by reference herein.

### Utility

The devices and methods of the invention, e.g., as described above, find use in a variety of different applications, e.g., applications where implantation of an annulosplasty device is desired. As such, the devices and methods of invention find use in the surgical treatment of heart valve disease, including cardiac valve dysfunction. More specifically, and as noted above, for patients who suffer from dysfunction of the mitral and/or tricuspid valve(s) of the heart, surgical repair of the valve (i.e., “valvuloplasty”) is a desirable alternative to valve replacement. Remodeling of the valve annulus (i.e., “annuloplasty”) is central to many reconstructive valvuloplasty procedures. Devices and methods described herein find use in such procedures. In such procedures, annuloplasty devices of the invention may be implanted to stabilize the annulus and to correct or prevent valvular insufficiency that may result from defect dysfunction of the valve annulus. The annuloplasty devices so implanted may maintain coaptation and valve integrity to prevent reverse flow while permitting good hemodynamics during forward flow. Annuloplasty procedures using devices as described herein may be performed not only to repair damaged or diseased annuli, but also in conjunction with other procedures, such as leaflet repair.

The disclosed devices and methods provide annuloplasty devices and methods to implant such devices in a time-efficient manner. More specifically, implantation of an annuloplasty device, including measuring the dimensions of the patient's heart valves and anchoring (e.g., suturing), an annuloplasty device within the heart, can be a time-consuming process. In addition, introducing and/or anchoring an annuloplasty device to cardiac tissue is often technically difficult and/or time-consuming when using minimally invasive procedures because of limitations in using 2-dimensional video for viewing the surgical field, limited exposure of the surgical field, and/or limited degrees of freedom when using standard thoracoscopic instrumentation.

By using the subject devices and methods, such as using tissue securing regions of annuloplasty devices in securing annuloplasty rings to a tissue site, (e.g., a cardiac valve), the total time that an implantation procedure takes can be reduced. The time of such a procedure can be reduced by eliminating or reducing the time required for suturing an annuloplasty device to a tissue site. A reduced time for a surgical process can help prevent fatigue in attending medical staff and can otherwise reduce risk to the patient.

Additionally, by applying the described devices and methods in securing annuloplasty rings to a tissue site, the total trauma incurred by a tissue at the site of implantation may be reduced. For example, by applying the disclosed devices and methods, the need for sutures an associated trauma in attaching an annuloplasty ring to a cardiac site can be reduced or eliminated.

Furthermore, by applying the disclosed devices and methods, such as using tissue securing regions of annuloplasty devices in securing annuloplasty rings to a tissue site, the securing process can be significantly simplified (e.g., made easier to understand) and/or made easier to or perform. For example, the process may be simplified by reducing or eliminating the amount of sutures (e.g., sutures within the heart) needed for implantation of the device. A process of annuloplasty device implantation may also be simplified by reducing the total steps performed using a 2-dimensional video for viewing the surgical field, while having a limited exposure of the surgical field, and/or while having limited degrees of freedom by using standard thoracoscopic instrumentation.

### Kits

Also provided are kits that at least include the subject devices and which may be used according to the subject methods. The subject kits at least include an annuloplasty device and a tissue securing device. Embodiments of the disclosed kits include annuloplasty devices having a body (e.g., an at least partially annular body) and one or more tissue securing regions integrated into and of differing composition from the body. Such tissue securing regions may be configured (e.g., shaped and/or sized) to receive a tissue securing device or a portion thereof. The disclosed kits, in various embodiments, include any of the embodiments of the annuloplasty devices described herein or any combinations thereof.

In certain embodiments, the disclosed kits include one or more tissue securing devices (e.g., a tissue securing device configured to be operably associated with a tissue securing region of an annuloplasty device of the kit). The disclosed kits, in various embodiments, include any of the embodiments of the tissue securing devices described herein or any combinations thereof. Certain embodiments of the kits disclosed herein include tissue securing devices which include an implantable body (e.g., a portion of one or more materials, such as any of the materials described herein) having a proximal deployable arm at a proximal end and/or a distal deployable arm at a distal end. In such embodiments, the proximal and distal deployable arms may, upon deployment, be in a non-coplanar (e.g., perpendicular and/or orthogonal) configuration. In various embodiments of the devices of the kits, the proximal and distal deployable arms each define a plane which has the thickness of each respective corresponding arm and which are perpendicular and/or orthogonal to one another. Embodiments of tissue securing devices of the disclosed kits include a body (e.g., an implantable body and/or a longitudinal body) having a first end and a second end and the first end and/or the second end is barbed (i.e., includes one or more barbs thereon). Certain embodiments of the disclosed kits include tissue securing devices which include a material that is a shape memory material (i.e., a material that is biased to return to its original form when it is deformed from its original form). In various aspects, the shape memory material of a tissue securing device may be any of the shape memory materials described herein. Tissue securing devices or characteristics thereof that may be utilized either wholly or partially in connection with the disclosed methods are provided by U.S. Pat. App. No. 61/831,454 and international application serial no. PCT/US2014/______ having Attorney Docket No. LCTHX-008WO and titled “TISSUE ANCHOR AND DEPLOYMENT DEVICE FOR SAME”, filed on Jun. 4, 2014; and U.S. Pat. No. 6,447,524; the disclosures of each which are incorporated by reference herein.

In addition, the kits may include a delivery device for the tissue securing device, e.g., a delivery gun, such as described in U.S. Pat. Nos. 6,425,900 and 6,447,524, as well as U.S. Patent Application No. 61/831,454 and international application serial no. PCT/US2014/______ having Attorney Docket No. LCTHX-008WO and titled “TISSUE ANCHOR AND DEPLOYMENT DEVICE FOR SAME”, filed on Jun. 4, 2014; the disclosures of which are herein incorporated by reference.

In certain embodiments, the kits which are disclosed herein include instructions, such as instructions for using devices. The instructions for using devices are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging etc.). In other embodiments, the instructions are present as an electronic storage data file present on a suitable computer readable storage medium, e.g., Portable Flash drive, CD-ROM, diskette, etc. The instructions may take any form, including complete instructions for how to use the device or as a website address with which instructions posted on the world wide web may be accessed. Any of the components may be present in containers or packaging, where two or more components may be present in the same container, e.g., as desired. In some instances, the containers/packaging are sterile, e.g., to maintain the sterility of the components of the kit, such as the components that are ultimately to be implanted into a patient.

In addition, embodiments of the disclosed kits or their components may be used according to any of the embodiments of the methods described herein or combinations thereof.

The following example is offered by way of illustration and not by way of limitation.

### EXPERIMENTAL

A patient is prepared for a mitral annuloplasty ring implantation procedure in a conventional manner. The patient is anesthetized using conventional anesthesia and anesthesiology procedures.

The patient undergoes an intraoperative transesophageal echocardiography to determine the responsible mechanisms for mitral dysfunction (e.g., mitral regurgitation). The intraoperative transesophageal echocardiography also serves as a baseline evaluation for assessing the quality of the repair, and for follow-up evaluation.

The patient's skin overlying the sternum and surrounding areas is swabbed with a conventional disinfecting solution. Next, the surgeon accesses the patient's thoracic cavity and approaches the heart through a median sternotomy. Aortic cannulation and cannulation of the superior and inferior vena cave are then performed by conventional means.

Next, the patient is placed on cardiopulmonary bypass in a conventional manner and the patient's heart is stopped from beating in a conventional manner. The surgeon then implants an annuloplasty ring device in the following manner: Superficial tissue overlying the Waterston's groove is dissected prior to incising the dilated left atrium to improve exposure of the mitral valve. After exposure of the mitral valve, the desired size of the annuloplasty device is determined by measuring the distance between the anterior and posterior comissures, the anterior leaflet height and/or the surface area of the anterior and posterior mitral leaflets.

An annuloplasty device as depicted in FIG. 1 is selected from a set of annuloplasty devices of the present invention based on the measurement. The annuloplasty device is then advanced into the left atrium using a delivery device and seated onto the annulus. Once the annuloplasty device is in the proper position, it is held in that position while an apparatus for deploying a tissue securing device is placed in an orientation to deploy a tissue securing device into a tissue securing region of the annuloplasty device. The apparatus for deploying a tissue securing device is then actuated to deploy a portion of a tissue securing device through a tissue securing region and into the tissue of the annulus below. When a portion of a tissue securing device is deployed through a tissue securing region into the tissue of the annulus below, a portion of the tissue securing device is retained on the upper surface of the annuloplasty device to hold the annuloplasty device against the underlying annulus.

The deploying apparatus is then re-oriented to deploy a tissue securing device into a second tissue securing region of the annuloplasty device and actuated to deploy a portion of a tissue securing device through the second tissue securing region and into the tissue of the annulus below. The process of re-orienting the deploying apparatus and deploying a portion of a tissue securing device through a tissue securing region into underlying tissue is repeated until the annuloplasty device is securely affixed to the annulus.

Post-implant valve competency can be assessed by filling and pressurizing the left ventricle with saline and observing the valve. The incisions are then closed and the patient weaned, or removed, from cardiopulmonary bypass. After weaning the patient from cardiopulmonary bypass, valve function is examined with transesophageal echocardiography or like means. The chest and skin incisions are then closed to complete the procedure.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. An annuloplasty device comprising:
an at least partially annular body; and
a tissue securing region integrated into and of differing composition from the body, where the tissue securing region is configured to receive a tissue securing device.

2. The annuloplasty device according to claim 1, wherein the annuloplasty device comprises two or more spaced apart tissue securing regions integrated into the body.

3. The annuloplasty device according to claim 2, wherein the annuloplasty device comprises three or more equidistantly spaced apart tissue securing regions integrated into the body.

4-8. (canceled)

9. The annuloplasty device according to claim 1, wherein the tissue securing region is marked.

10. The annuloplasty device according to claim 9, wherein the tissue securing region is marked by having a color that differs from the color of the body immediately adjacent the tissue securing region.

11. The annuloplasty device according to claim 1, wherein the annuloplasty device is configured to be implanted at a cardiac valve site.

12. The annuloplasty device according to claim 10, wherein the cardiac valve site is a mitral valve site.

13. The annuloplasty device according to claim 1, wherein the annuloplasty device is shaped as a ring.

14-15. (canceled)

16. The annuloplasty device according to claim 1, wherein the annuloplasty device is flexible.

17-19. (canceled)

20. The annuloplasty device according to claim 1, wherein body is a ring comprising a first material and the tissue securing region comprises a void associated with the body, wherein the void is filled with a second material that is more compliant than the first material.

21. The annuloplasty device according to claim 20, wherein the device further comprises a coating component which coats at least a portion of body.

22. The annuloplasty device according to claim 21, wherein the coating component comprises the second material.

23. The annuloplasty device according to claim 20, wherein the device further comprises an outer cover comprising a third material.

24. The annuloplasty device according to claim 20, wherein the first material comprises a shape memory material.

25. The annuloplasty device according to claim 24, wherein the shape memory material is a metal alloy.

26. The annuloplasty device according to claim 25, wherein the metal alloy is a nickel alloy.

27-28. (canceled)

29. The annuloplasty device according to claim 20, wherein the second material is a silicone.

30. The annuloplasty device according to claim 23, wherein the third material comprises a fabric.

31-32. (canceled)

33. A method comprising implanting an annulopasty device at a tissue site, the method comprising:
(a) positioning an annuloplasty device at the tissue site, wherein the annuloplasty device comprises:
(i) an at least partially annular body; and
(ii) a tissue securing region of integrated into and of differing composition from the body, where the tissue securing region is configured to receive a tissue securing device; and
(b) engaging tissue of the tissue site with a tissue securing device operably associated with the tissue securing region in a manner sufficient to stably implant the annuloplasty device at the tissue site.

34-47. (canceled)

48. A kit comprising:
(a) an annuloplasty device comprising:
(i) an at least partially annular body; and
(ii) a tissue securing region integrated into and of differing composition from the body, where the tissue securing region is configured to receive a tissue securing device; and
(b) a tissue securing device configured to be operably associated with the tissue securing region.

49-55. (canceled)
